(19) United States
(12) Patent Application Publication
(10) Pub. No.: US 2026/0216528 A1
(43) Pub. Date: Jul. 30, 2026

(54) Inflatable Light Therapy Apparatus

(71) Applicants: Joseph Yi, Lynnwood, WA (US); Daniel Yi, Lynnwood, WA (US)

(72) Inventors: Joseph Yi, Lynnwood, WA (US); Daniel Yi, Lynnwood, WA (US)

(73) Assignee: Joseph Yi LLC, Lynnwood, WA (US)

(21) Appl. No.: 19/464,553

(22) Filed: Jan. 29, 2026

Related U.S. Application Data
(60) Provisional application No. 63/751,386, filed on Jan. 30, 2025.

(51) Int. Cl. A61N 5/06

(52) U.S. Cl. CPC A61N 5/06; A61N 2005/0633; A61N 2005/0634; A61N 2005/0638; A61N 2005/0639; A61N 2005/0652; A61N 2005/0659; A61N 2005/0661; A61N 2005/0663

(57) ABSTRACT

Described herein is a light therapy apparatus comprising a base member including a plurality of light emitting elements dispersed on a top surface, a shell member removably coupled to the base member such that the shell member is rotatable between an unfolded position and a folded position, wherein the shell member includes an interior surface having a plurality of light emitting elements, wherein the base member and the shell member are each inflatable, wherein when in a deflated state and the folded position the light emitting elements of the shell member are planar and in a stacked configuration with the light emitting elements of the base member, and wherein when in an inflated state the shell member forms a dome shape such that the light emitting elements of the shell member are angularly offset relative to the light emitting elements of the base member.

### CROSS-REFERENCE TO RELATED APPLICATIONS

This application claims the benefit of U.S. Provisional Patent Application No. 63/751,386, filed on Jan. 30, 2025, which is hereby incorporated by reference in its entirety.

### FIELD OF THE INVENTION

The present invention relates to a light therapy apparatus, and more particularly to an inflatable light therapy apparatus.

### BACKGROUND OF THE INVENTION

Light therapy has emerged as a widely recognized treatment modality for promoting healing, reducing inflammation, and improving cellular function. Typically, these therapeutic techniques utilize low-level wavelengths of red and near-infrared light to penetrate the skin and target tissues, stimulating mitochondrial activity and enhancing ATP production. Current devices range from large panels for whole-body treatment to handheld devices for localized application.

However, traditional existing systems face challenges such as limited portability, inconsistent light distribution, singular purpose products, and lack of customizable settings tailored to specific therapeutic needs.

### SUMMARY OF THE INVENTION

The present invention provides a light therapy apparatus including a base member configured to define a resting platform for receiving a user. In some embodiments, the base member includes a plurality of light emitting elements dispersed on a top surface of the resting platform. In some embodiments, the light therapy apparatus includes a shell member removably coupled to the base member such that the shell member is rotatable between an unfolded position and a folded position. In some embodiments, the shell member includes an interior surface having a plurality of light emitting elements. In some embodiments, the base member and the shell member include an internal volume configured for inflation and configured to transition from a deflated state to an inflated state. In some embodiments, when the base member and the shell member are in the deflated state and the shell member is in the folded position, the plurality of light emitting elements of the shell member are planar and in a stacked configuration with the plurality of light emitting elements of the base member. In some embodiments, when the base member and the shell member are in the inflated state, the shell member forms a dome shape such that the plurality of light emitting elements of the shell member are angularly offset relative to the plurality of light emitting elements of the base member.

In some embodiments, the plurality of light emitting elements include a first portion of light emitting elements that emit visible wavelengths and a second portion of the light emitting elements that emit non-visible wavelengths. In some embodiments, the first portion and the second portion of the plurality of light emitting elements are independent of each other such that both portions can simultaneously emit different wavelengths.

In some embodiments, the first portion of light emitting elements are LEDs, and the second portion of light emitting elements are infrared emitting elements.

In some embodiments, the number of the plurality of light emitting elements dispersed within the light therapy apparatus is greater than 100.

In some embodiments, the LEDs emit wavelengths between 380 nm-780 nm, and the infrared emitting elements emit wavelengths between 780 nm-1000 nm.

In some embodiments, the shell member includes an exterior surface having a display panel configured to display data from the group consisting of temperature, timing, and light therapy treatment modes.

In some embodiments, the resting platform includes one or more speakers. In some embodiments, the one or more speakers wirelessly connect with the display panel for outputting information displayed on the display panel in audio format.

In some embodiments, the shell member includes one or more sections. In some embodiments, at least one of the one or more sections is transparent and configured for visibility during operation of the light therapy apparatus.

In some embodiments, when in the inflated state, the light therapy apparatus includes a therapy zone positioned between the resting platform of the base member and the interior surface of the shell member. In some embodiments, the therapy zone is configured for optimal light emittance during operation of the light therapy apparatus.

In another embodiment, the light therapy apparatus includes a base member including a top surface. In some embodiments, the top surface includes a plurality of light emitting elements disposed on the top surface. In some embodiments, the light therapy apparatus includes a shell member hingedly coupled to the base member. In some embodiments, the shell member includes one or more viewing sections and one or more light therapy sections. In some embodiments, the one or more viewing sections are transparent. In some embodiments, the one or more light therapy sections include a plurality of light emitting elements disposed on an interior surface of the shell member.

In some embodiments, the base member and the shell member include an internal volume configured for inflation and configured to transition from a deflated state to an inflated state.

In some embodiments, when the base member and the shell member are in the inflated state, the shell member forms a dome shape such that the plurality of light emitting elements of the shell member are angularly offset relative to the plurality of light emitting elements of the base member.

In another embodiment, the light therapy apparatus includes an inflatable base member including a plurality of light emitting elements disposed on a top surface of the base member, and an inflatable shell member hingedly attached to the base member along a fold line. In some embodiments, the shell member includes a plurality of light emitting elements disposed on an interior surface of the shell member. In some embodiments, the light therapy apparatus is configured to transition between a folded position and an unfolded position. In some embodiments, when the light therapy apparatus is in the folded position the shell member is disposed on top of the base member. In some embodiments, when the light therapy apparatus is in the unfolded position the shell member is adjacent to the base member at the fold line. In some embodiments, the light therapy apparatus is configured to transition between an inflated state and a deflated state. In some embodiments, when in the folded position and the deflated state the light therapy apparatus is compactable into a portable configuration.

In some embodiments, the light therapy apparatus includes a bottom surface having a foldable handbar configured for facilitating manual repositioning of the light therapy apparatus without requiring lifting of the light therapy apparatus.

In some embodiments, the bottom surface of the light therapy apparatus includes one or more tensioning elements and one or more releasable coupling elements each configured for securing the light therapy apparatus into the portable configuration.

In some embodiments, the bottom surface includes one or more friction members and one or more fastening members each configured for increasing friction between the light therapy apparatus and an underlying surface.

In some embodiments, when in the inflated state, the base member includes a first width and the shell member includes a second width, and the first width of the base member is greater than the second width of the shell member.

In some embodiments, when in the inflated state, the shell member includes a dome shape, and the base member includes a flat profile, and the dome shaped shell member is fully disposed on top of the flat profiled base member.

In some embodiments, the fold line is disposed on the top surface of the base member.

In some embodiments, the shell member is separable from the base member.

### BRIEF DESCRIPTION OF THE DRAWINGS

The features of the exemplary embodiments of the present invention will be described with reference to the following drawings, where like elements are labeled similarly, and in which:

FIG. 1 is an isometric view of a light therapy apparatus in an unfolded position and inflated state according to the present disclosure;

FIG. 2 is an isometric view of the light therapy apparatus of FIG. 1 in a folded position and inflated state according to the present disclosure;

FIG. 3 is a side view of the light therapy apparatus of FIG. 1 according to the present disclosure;

FIG. 4 is a top-down view of the light therapy apparatus of FIG. 1 in the folded position according to the present disclosure;

FIG. 5 is a bottom view of the light therapy apparatus of FIG. 1 according to the present disclosure;

FIG. 6 is a top-down view of the light therapy apparatus of FIG. 1 in the unfolded position according to the present disclosure; and

FIG. 7 is an isometric view of the light therapy apparatus of FIG. 1 in a portable configuration according to the present disclosure.

All drawings are schematic and not necessarily to scale. Parts given a reference numerical designation in one figure may be considered to be the same parts where they appear in other figures without a numerical designation for brevity unless specifically labeled with a different part number and described herein.

### DETAILED DESCRIPTION OF THE EMBODIMENTS

The features and benefits of the invention are illustrated and described herein by reference to exemplary embodiments. This description of exemplary embodiments is intended to be read in connection with the accompanying drawings, which are to be considered part of the entire written description. Accordingly, the disclosure expressly should not be limited to such exemplary embodiments illustrating some possible non-limiting combination of features that may exist alone or in other combinations of features.

In the description of embodiments disclosed herein, any reference to direction or orientation is merely intended for convenience of description and is not intended in any way to limit the scope of the present invention. Relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise.

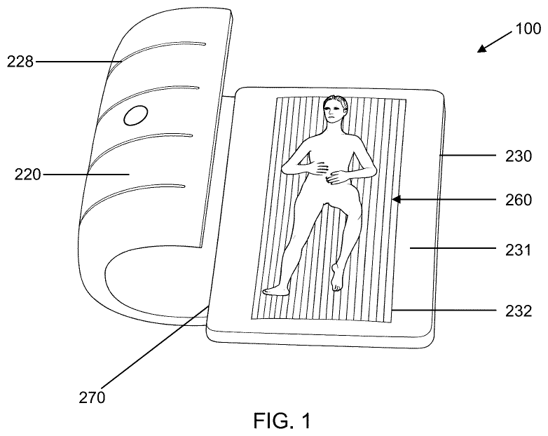

FIG. 1 depicts an exemplary embodiment of a light therapy apparatus 100 for providing portable light therapy application to users to help promote healthy skincare routines and to minimize acne, wrinkles, and other skincare conditions. Unlike traditional light therapy devices which are rigid and bulky devices such as masks, hats, and chambers, the light therapy apparatus 100 of the present disclosure is a flexible, inflatable apparatus that can transition between multiple positions and states to accommodate various use cases. The light therapy apparatus 100 of the present disclosure utilizes an inflatable design to create a dome shape that allows light emitting elements to radially emit light therapy to the user such that the light being emitted converges to a central portion of the light therapy apparatus 100 where the user is located. The light therapy apparatus 100 includes a plurality of light emitting elements 260 that are capable of emitting a wide range of wavelengths, including but not limited to red light for reducing wrinkles, fine lines, and promoting collagen growth; white light for enhancing nutrient absorption; yellow light for brightening skin complexion; green light for smoothing skin texture; cyan light for reducing stress and sun damage; blue light for reducing acne, redness, and calming irritated skin; purple light for reducing swelling; and infrared light for hair growth, improving skin appearance, reducing wrinkles, and reducing sun damage.

As shown in FIG. 1, the light therapy apparatus 100 includes a shell member 220 and a base member 230. In some embodiments, the shell member 220 is an upper portion of the light therapy apparatus 100 that can arch over a user during treatment. In some embodiments, the base member 230 is a lower portion of the light therapy apparatus 100 upon which a user rests during treatment.

In some embodiments, the light therapy apparatus 100 is configured to transition between an inflated state and a deflated state. In some embodiments, the base member 230 and the shell member 220 include an internal volume configured for inflation. A person having ordinary skill in the art would understand that the light therapy apparatus 100 can be positioned into any combination of folded position, unfolded position, inflated state and deflated state. For example, the light therapy apparatus 100 may be in the unfolded position and inflated state, (as shown in FIG. 1) configured for single sided light therapy treatment provided only by the plurality of light emitting elements dispersed on the top surface of the resting platform. In other embodiments, the light therapy apparatus 100 may be in the unfolded position and deflated state for use as a traditional blanket with ambient light therapy.

In some embodiments, the shell member 220 and the base member 230 are connected along a fold line 270. In some embodiments, the fold line 270 enables the light therapy apparatus 100 to hingedly transition between an unfolded position and a folded position. In some embodiments, to transition from the folded position to the unfolded position, the shell member 220 may extend upwardly and outwardly away from the base member 230 from where they are connected along the fold line 270. In some embodiments, the fold line 270 may be formed of a bonded material, a sewn seam, a welded joint, or a flexible hinge material that allows repeated folding and unfolding with minimal degradation over time. In some embodiments, a hinged connection of the fold line 270 provides functional advantages over a permanently fixed or fully separate two-piece design, such as allowing the light therapy apparatus 100 to open and close in a clamshell-like manner which enables a user to easily enter and exit the therapy zone without requiring assistance or complex manipulation of the apparatus. A user can simply lift the shell member 220 away from the base member 230, position themselves on the resting platform 231, and then lower the shell member 220 over their body to begin treatment. In some embodiments, the fold line 270 further permits a caregiver or partner to open the shell member 220 during a treatment session to observe the user, adjust positioning, provide water or comfort items, or address any concerns without fully disrupting the treatment. In some embodiments, the ability to fully open the light therapy apparatus 100 into the unfolded position also facilitates thorough cleaning and inspection of interior surfaces between treatment sessions, promoting hygiene and allowing users to verify that the apparatus is functioning properly before each use. In some embodiments, the shell member 220 is removably coupled to the base member 230. In some embodiments, separability of the shell member 220 from the base member 230 enables replacement of either component if damaged without requiring replacement of the entire apparatus, allows each member to be cleaned and maintained independently, and permits users to swap shell members or base members having different light emitting configurations for varied treatment options. For example, a user might own multiple shell members configured for different treatment purposes and attach the appropriate shell member to the base member 230 depending on their current skincare needs. Although the present disclosure describes a light therapy apparatus having two primary members connected by a single fold line 270, a person having ordinary skill in the art would understand that the light therapy apparatus 100 may include any number of segments with multiple fold lines while still providing similar function.

In some embodiments, the base member 230 includes a resting platform 231 for receiving a user. In some embodiments, the resting platform 231 provides a cushioned surface that supports a user's body during treatment sessions. In some embodiments, the resting platform 231 includes a top surface 232. In some embodiments, the plurality of light emitting elements 260 are dispersed on the top surface 232 of the resting platform 231. In some embodiments, the top surface 232 is centrally disposed within the resting platform 231, such that the plurality of light emitting elements 260 do not extend entirely across the full width of the resting platform 231. In some embodiments, central positioning of the plurality of light emitting elements 260 on the resting platform 231 creates a peripheral border region around the top surface 232 which may include padding that provides additional comfort for users' arms, shoulders, and legs extending beyond the primary treatment area, or which may serve to house internal wiring, control circuitry, or structural reinforcement elements that support the resting platform 231 without interfering with the plurality of light emitting elements 260. In some embodiments, the border region further creates a natural transition zone that helps users orient themselves correctly on the resting platform 231, as they can feel the edges of the top surface 232 under their body when laying on their back, and center their body accordingly.

In some embodiments, as shown for example in FIG. 1, the shell member 220 includes one or more viewing sections 228. In some embodiments, the one or more viewing sections 228 are transparent and configured for visibility during operation of the light therapy apparatus 100. In some embodiments, the one or more viewing sections 228 allow a user to see outside of the light therapy apparatus 100 while undergoing treatment, which may reduce feelings of claustrophobia or confinement that some users experience when enclosed within treatment devices. In some embodiments, visibility provided by the one or more viewing sections 228 allows the user to monitor their surroundings, watch television or other media, read, interact with family members or caregivers, or simply observe the room during treatment. Visibility can make treatment sessions feel less isolating and may encourage users to complete full treatment durations rather than ending sessions early due to discomfort. In some embodiments, the shell member 220 simultaneously provides therapeutic light exposure through light therapy sections while maintaining user visibility through the one or more viewing sections 228.

In some embodiments, the fold line 270 is disposed on top of the base member 230 rather than underneath the base member 230 to protect the fold line 270 from wear, abrasion, and damage when the light therapy apparatus 100 rests on underlying surfaces during use. In some embodiments, such positioning of the fold line 270 also creates a slimmer profile when the apparatus is in the folded position, as the fold line 270 does not add bulk or create uneven pressure points on the underside of the apparatus.

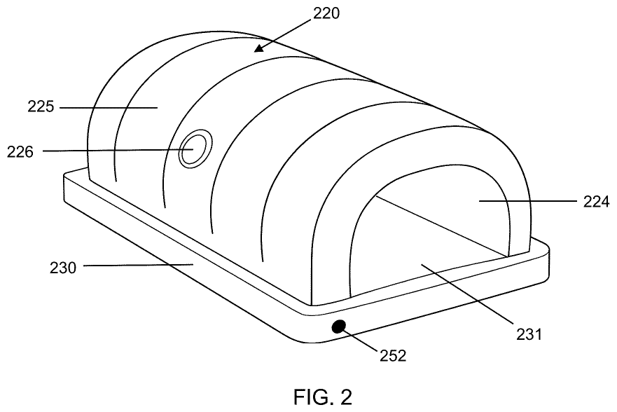

As shown in FIG. 2, the light therapy apparatus 100 may be configured to be in the folded position and the inflated state. In some embodiments, when in the folded position and the inflated state, the shell member 220 is positioned overtop of the base member 230 for concentrating light therapy on a user positioned within, which facilitates full body light therapy treatment. In some embodiments, the shell member 220 includes an exterior surface 225 that faces outward and away from the user during treatment. In some embodiments, the exterior surface 225 includes a display panel 226. In some embodiments, the display panel 226 is configured to display operational information such as temperature, elapsed time, remaining time, selected light wavelengths, active treatment modes, power level, battery status, and error notifications. In some embodiments, as the display panel 226 is disposed on the exterior surface 225 of the shell member 220, the display panel 226 faces away from the user during treatment. In some embodiments, outward-facing positioning of the display panel 226 allows a therapy facilitator, caregiver, family member, others to monitor treatment information and settings. For example, a therapy facilitator can observe the display panel 226 during treatment to ensure proper operation without disturbing the user, and can adjust treatment parameters if needed. In some embodiments, the display panel 226 includes touch-sensitive controls for adjusting treatment parameters directly on the apparatus. In some embodiments, the display panel 226 is an OLED touchscreen panel.

In some embodiments, the base member 230 further includes an inflation valve 252. In some embodiments, the inflation valve 252 is disposed on an outer edge of the base member 230. In some embodiments, the inflation valve 252 is configured for inflating both the base member 230 and the shell member 220 from the deflated state to the inflated state. In some embodiments, the inflation valve 252 is configured for deflating both the base member 230 and the shell member 220 from the inflated state to the deflated state. In some embodiments, the shell member 220 and the base member 230 share a common internal air chamber such that inflating or deflating through the inflation valve 252 simultaneously inflates or deflates both members through a single valve operation. In some embodiments, the inflation valve 252 is fluidly connected to separate air chambers within the shell member 220 and the base member 230 through internal passages. In some embodiments, the inflation valve 252 is compatible with standard air pumps, electric inflators, or manual inflation. In some embodiments, the inflation valve 252 includes a release mechanism for controlled deflation when the user wishes to transition the light therapy apparatus 100 from the inflated state to the deflated state for storage or transport.

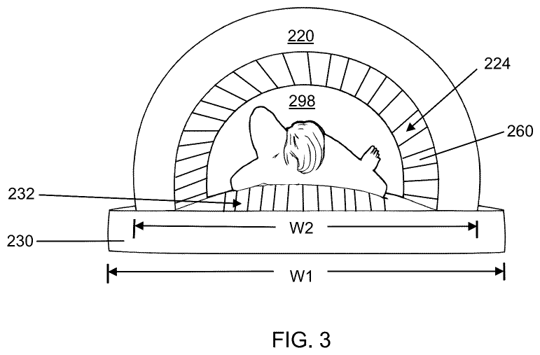

As shown in FIG. 3, the plurality of light emitting elements 260 are dispersed on an interior surface 224 of the shell member 220 and the top surface 232 of the resting platform 231. In some embodiments, as shown for example in FIG. 3, when the light therapy apparatus 100 is in the folded position and the inflated state, the interior surface 224 faces inward toward the user during treatment and a therapy zone 298 is created between the interior surface 224 of the shell member 220 and the resting platform 231 of the base member 230. In some embodiments, the therapy zone 298 is an enclosed space configured for positioning of a user, during operation of the light therapy apparatus 100, to receive optimal light emittance during operation of the light therapy apparatus 100. In some embodiments, the dome shape of the shell member 220 allows the plurality of light emitting elements 260 dispersed on the interior surface 224 to radially converge light toward a central region within the therapy zone 298 where a user is located. In some embodiments, radial convergence of light maximizes the amount of light therapy received by the user and reduces wasted light that may otherwise miss the user in flat-panel configurations. In some embodiments, the plurality of light emitting elements 260 dispersed on the top surface 232 of the resting platform 231 further contributes to treatment by emitting light upward toward the user's back, legs, or other body regions on the resting platform 231. In some embodiments, a combination of downward-angled light from the interior surface 224 and upward-directed light from the top surface 232 provides comprehensive light exposure within the therapy zone 298.

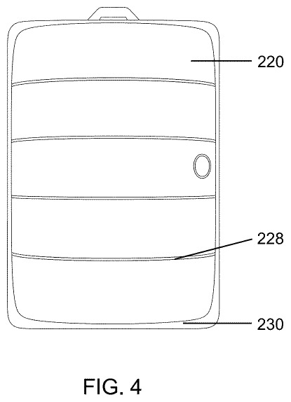

In some embodiments, as shown for example in FIG. 3, the base member 230 includes a first width W1 and the shell member 220 includes a second width W2. In some embodiments, the first width W1 of the base member 230 is greater than the second width W2 of the shell member 220. In some embodiments, the dome shaped shell member 220 is fully disposed on top of the flat profiled base member 230, such that the perimeter of the shell member 220 does not extend beyond the perimeter of the base member 230 at any point around the light therapy apparatus 100 (as shown in FIG. 4). In some embodiments, the wider base member 230 creates a stable foundation that fully supports the shell member 220, preventing the shell member 220 from tipping, shifting, or rolling during use even if the user moves or adjusts position during treatment. In some embodiments, a peripheral area of the resting platform 231 that extends beyond the shell member 220 provides additional usable space where a user can place personal items such as a phone, remote control, water bottle, or reading materials within arm's reach during treatment.

In some embodiments, the plurality of light emitting elements 260 include a first portion of light emitting elements that emit visible wavelengths and a second portion of light emitting elements that emit non-visible wavelengths. In some embodiments, the first portion and the second portion of the plurality of light emitting elements 260 are independent of each other such that both portions can simultaneously emit different wavelengths. In some embodiments, independent portions of the plurality of light emitting elements 260 allows the light therapy apparatus 100 to provide combination treatments where visible light addresses surface-level skin concerns while infrared light penetrates deeper into tissue for therapeutic benefits. In some embodiments, the first portion of light emitting elements are LEDs that are capable of emitting visible wavelengths within the wavelength range of 380 nm to 780 nm. In some embodiments, the second portion of light emitting elements are infrared emitting elements that are capable of emitting infrared light within the wavelength range of 780 nm to 1000 nm. In some embodiments, the number of the plurality of light emitting elements 260 dispersed within the light therapy apparatus 100 is greater than 100. A person having ordinary skill in the art would understand that the plurality of light emitting elements 260 can be configured to emit any wavelength within these ranges to provide specific benefits to the user based upon the desired treatment, and that the number of light emitting elements may vary depending on the size of the apparatus and the intensity of treatment desired.

In some embodiments, the light therapy apparatus 100 controls the wavelength being emitted from the plurality of light emitting elements 260 based upon the desired treatment for the user. In some embodiments, the present disclosure configures the plurality of light emitting elements 260 to emit the following light treatments at the following wavelengths: purple light treatment at approximately 390 nm for reducing facial swelling; blue light treatment at approximately 415 nm for eliminating acne-causing bacteria; cyan light treatment at approximately 490 nm for reducing stress and sun damage; green light treatment at approximately 525 nm for evening out skin texture; yellow light treatment at approximately 590 nm for brightening skin complexion; red light treatment at approximately 630 nm for reducing wrinkles and increasing collagen production; and white light through a combination of wavelengths for enhancing nutrient absorption. In some embodiments, the light therapy apparatus 100 allows the user to select multiple wavelengths simultaneously for combination treatments. In some embodiments, the light therapy apparatus 100 includes preset treatment modes that automatically cycle through different wavelengths during a single session, allowing users to receive comprehensive treatment without manually adjusting settings.

FIG. 4 depicts the light therapy apparatus 100 in a top down view in the folded position and the inflated state, illustrating the dome shaped shell member 220 disposed on the flat profiled base member 230. In some embodiments, as shown in FIG. 4, the one or more viewing sections 228 extend along the shell member 220 to provide the user with a wide field of view. In some embodiments, an elongated shape of the one or more viewing sections 228 allows the user to see a substantial portion of the surrounding room without turning their head, enhancing comfort during treatment sessions.

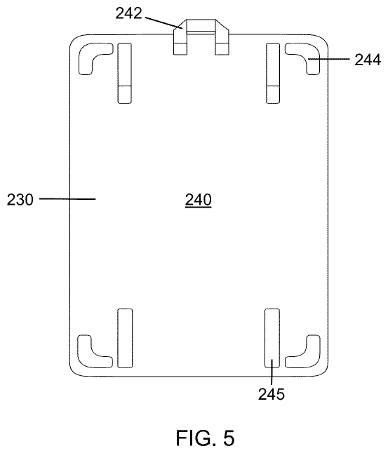

As shown in FIG. 5, the base member 230 includes a bottom surface 240. In some embodiments, the bottom surface 240 faces downward and contacts an underlying surface such as a floor, bed, massage table, or treatment platform during use. In some embodiments, the bottom surface 240 includes a foldable handbar 242. In some embodiments, the foldable handbar 242 is configured for facilitating manual repositioning of the light therapy apparatus 100 without requiring lifting of the light therapy apparatus 100. A user or caregiver can grasp the foldable handbar 242 and drag or slide the light therapy apparatus 100 to a desired location, which is particularly useful when the apparatus is inflated and cumbersome to lift. In some embodiments, the foldable handbar 242 is stored and hidden in a folder or compartment, allowing the light therapy apparatus 100 to rest evenly on underlying surfaces without the foldable handbar 242 creating an uneven gap, wobble, or pressure point beneath the apparatus.

In some embodiments, the bottom surface 240 of the base member 230 includes one or more friction members 244 and one or more fastening members 245. In some embodiments, the one or more friction members 244 and the one or more fastening members 245 are each configured for increasing friction between the light therapy apparatus 100 and an underlying surface. In some embodiments, increased friction prevents the light therapy apparatus 100 from sliding, shifting, or migrating during use, which could otherwise occur if the user moves or adjusts position during treatment. In some embodiments, the one or more friction members 244 are rubberized pads, silicone grips, or textured surfaces positioned at strategic locations along the bottom surface 240 that grip the underlying surface through mechanical friction without requiring permanent attachment. In some embodiments, the one or more fastening members 245 are hook and loop fasteners that releasably attach to compatible surfaces, straps, or accessories. In some embodiments, the one or more fastening members 245 enable the light therapy apparatus 100 to be secured to a massage table, bed frame, or dedicated treatment platform equipped with corresponding fastening strips, providing additional stability for users who may move significantly during treatment sessions.

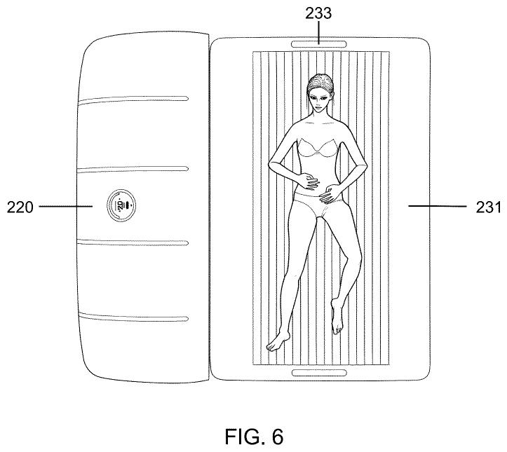

As shown in FIG. 6, the resting platform 231 of the base member 230 includes one or more speakers 233. In some embodiments, the one or more speakers 233 are positioned on the resting platform 231 in locations that direct audio toward a user lying on the base member 230. In some embodiments, the one or more speakers 233 are Bluetooth-enabled speakers configured to wirelessly connect with external devices such as smartphones, tablets, or media players. In some embodiments, the one or more speakers 233 allow the user to play music, soothing nature sounds, guided meditation audio, audiobooks, podcasts, or other audio content during treatment sessions to enhance relaxation and enjoyment. In some embodiments, the one or more speakers 233 also enable the user to take phone calls while using the light therapy apparatus 100, allowing the user to remain connected and productive during treatment. In some embodiments, as the display panel 226 is disposed on the exterior surface 225 of the shell member 220, a user positioned within the therapy zone 298 cannot view the display panel 226 during treatment without exiting the therapy zone 298. In some embodiments, the one or more speakers 233 output information displayed on the display panel 226 in audio format. In some embodiments, the one or more speakers 233 announce treatment time remaining at regular intervals, alert the user to temperature changes, notify the user of treatment mode transitions, warn the user of any errors or malfunctions requiring attention, and announce when treatment is complete. In some embodiments, audio feedback allows the user to remain informed about treatment status while keeping their eyes closed and body relaxed, avoiding the need to interrupt a treatment session to visually check the display panel 226.

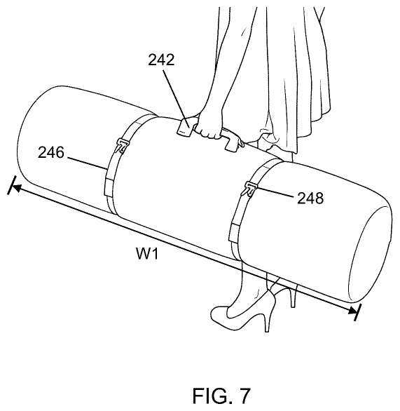

As shown in FIG. 7, when in the folded position and the deflated state, the light therapy apparatus 100 is compactable into a portable configuration. In some embodiments, to achieve the portable configuration, a user deflates the light therapy apparatus 100, folds the shell member 220 onto the base member 230 into the folded position, and then rolls or folds the light therapy apparatus 100 along the first width W1 of the base member 230 to form a compact bundle suitable for transport or storage. In some embodiments, the portable configuration of the light therapy apparatus 100 forms a tubular shape.

In some embodiments, the light therapy apparatus 100 includes one or more tensioning elements 246 and one or more releasable coupling elements 248, each configured for securing the light therapy apparatus 100 in the portable configuration. In some embodiments, the one or more tensioning elements 246 are straps, elastic bands, or webbing that wrap around the circumference of the light therapy apparatus 100 when in the portable configuration to keep the apparatus tightly compacted and prevent it from unraveling. In some embodiments, the one or more releasable coupling elements 248 are buckles, clips, snaps, or closures that secure the one or more tensioning elements 246 in place. In some embodiments, the combination of the one or more tensioning elements 246 and the one or more releasable coupling elements 248 maintains the compact portable configuration during transport and storage, while allowing quick release when the user is ready to deploy the light therapy apparatus 100 for use. In some embodiments, the foldable handbar 242 remains accessible when the light therapy apparatus 100 is in the portable configuration, allowing the user to carry the compacted apparatus by the foldable handbar 242. In some embodiments, the portable configuration allows the light therapy apparatus 100 to fit in closets, under beds, in vehicle trunks, or in dedicated storage bags for easy transport to different locations.

In some embodiments, the light therapy apparatus 100 is composed of soft, comfortable materials suitable for extended contact with a user's skin. In some embodiments, the light therapy apparatus 100 is composed of fleece material to provide comfort and warmth to users. In other embodiments, the light therapy apparatus 100 is composed of cotton, polyester, bamboo, microfiber, vinyl, or any combination thereof. In some embodiments, the one or more viewing sections 228 are composed of a transparent thermoplastic elastomer such as transparent thermoplastic polyurethane (TPU). In some embodiments, the one or more viewing sections 228 are UV-resistant to prevent degradation from prolonged exposure to the plurality of light emitting elements 260. In some embodiments, the foldable handbar 242 is composed of nylon webbing, reinforced fabric, or other durable materials capable of withstanding repeated grasping and pulling forces. A person having ordinary skill in the art would understand that the light therapy apparatus 100 and its components may be composed of any suitable materials without departing from the spirit or essential characteristics of the present disclosure.

While the foregoing description and drawings represent exemplary embodiments of the present disclosure, it will be understood that various additions, modifications and substitutions may be made therein without departing from the spirit and scope and range of equivalents of the accompanying claims. In particular, it will be clear to those skilled in the art that the present invention may be embodied in other forms, structures, arrangements, proportions, sizes, and with other elements, materials, and components, without departing from the spirit or essential characteristics thereof. In addition, numerous variations in the methods/processes described herein may be made within the scope of the present disclosure. One skilled in the art will further appreciate that the embodiments may be used with many modifications of structure, arrangement, proportions, sizes, materials, and components and otherwise, used in the practice of the disclosure, which are particularly adapted to specific environments and operative requirements without departing from the principles described herein. The presently disclosed embodiments are therefore to be considered in all respects as illustrative and not restrictive. The appended claims should be construed broadly, to include other variants and embodiments of the disclosure, which may be made by those skilled in the art without departing from the scope and range of equivalents.

What is claimed is:

1. A light therapy apparatus comprising:
a base member configured to define a resting platform for receiving a user, and wherein the base member includes a plurality of light emitting elements dispersed on a top surface of the resting platform;
a shell member removably coupled to the base member such that the shell member is rotatable between an unfolded position and a folded position, and wherein the shell member includes an interior surface having a plurality of light emitting elements;
wherein the base member and the shell member include an internal volume configured for inflation and configured to transition from a deflated state to an inflated state;
wherein when the base member and the shell member are in the deflated state and the shell member is in the folded position, the plurality of light emitting elements of the shell member are planar and in a stacked configuration with the plurality of light emitting elements of the base member; and
wherein when the base member and the shell member are in the inflated state, the shell member forms a dome shape such that the plurality of light emitting elements of the shell member are angularly offset relative to the plurality of light emitting elements of the base member.

2. The light therapy apparatus of claim 1, wherein the plurality of light emitting elements include a first portion of light emitting elements that emit visible wavelengths and a second portion of the plurality of light emitting elements that emit non-visible wavelengths, and wherein the first portion and the second portion of the plurality of light emitting elements are independent of each other such that both portions can simultaneously emit different wavelengths.

3. The light therapy apparatus of claim 2, wherein the first portion of light emitting elements are LEDs, and wherein the second portion of light emitting elements are infrared emitting elements.

4. The light therapy apparatus of claim 1, wherein the number of the plurality of light emitting elements dispersed within the light therapy apparatus is greater than 100.

5. The light therapy apparatus of claim 3, wherein the LEDs emit wavelengths between 380 nm -780 nm, and wherein the infrared emitting elements emit wavelengths between 780 nm -1000 nm.

6. The light therapy apparatus of claim 1, wherein the shell member includes an exterior surface having a display panel configured to display data from the group consisting of temperature, timing, and light therapy treatment modes.

7. The light therapy apparatus of claim 6, wherein the resting platform includes one or more speakers, and wherein the one or more speakers wirelessly connect with the display panel for outputting information displayed on the display panel in audio format.

8. The light therapy apparatus of claim 1, wherein the shell member includes one or more sections, and wherein at least one of the one or more sections is transparent and configured for visibility during operation of the light therapy apparatus.

9. The light therapy apparatus of claim 1, wherein when in the inflated state, the light therapy apparatus includes a therapy zone positioned between the resting platform of the base member and the interior surface of the shell member, and wherein the therapy zone is configured for concentrated light emittance during operation of the light therapy apparatus.

10. A light therapy apparatus comprising:
a base member including a top surface, wherein the top surface includes a plurality of light emitting elements disposed on the top surface;
a shell member hingedly coupled to the base member, wherein the shell member includes one or more viewing sections and one or more light therapy sections;
wherein the one or more viewing sections are transparent; and
wherein the one or more light therapy sections include a plurality of light emitting elements disposed on an interior surface of the shell member.

11. The light therapy apparatus of claim 10, wherein the base member and shell member include an internal volume configured for inflation and configured to transition from a deflated state to an inflated state.

12. The light therapy apparatus of claim 11, wherein when the base member and shell member are in the inflated state, the shell member forms a dome shape such that the plurality of light emitting elements of the shell member are angularly offset relative to the plurality of light emitting elements of the base member.

13. A light therapy apparatus comprising:
an inflatable base member including a plurality of light emitting elements disposed on a top surface of the base member;
an inflatable shell member hingedly attached to the base member along a fold line;
wherein the shell member includes a plurality of light emitting elements disposed on an interior surface of the shell member;
wherein the light therapy apparatus is configured to transition between a folded position and an unfolded position;
wherein when the light therapy apparatus is in the folded position the shell member is disposed on top of the base member;
wherein when the light therapy apparatus is in the unfolded position the shell member is adjacent to the base member at the fold line;
wherein the light therapy apparatus is configured to transition between an inflated state and a deflated state; and
wherein when in the folded position and the deflated state the light therapy apparatus is compactable into a portable configuration.

14. The light therapy apparatus of claim 13, wherein the light therapy apparatus includes a bottom surface having a foldable handbar configured for facilitating manual repositioning of the light therapy apparatus without requiring lifting of the light therapy apparatus.

15. The light therapy apparatus of claim 14, wherein the bottom surface of the light therapy apparatus includes one or more tensioning elements and one or more releasable coupling elements each configured for securing the light therapy apparatus into the portable configuration.

16. The light therapy apparatus of claim 14, wherein the bottom surface includes one or more friction members and one or more fastening members each configured for increasing friction between the light therapy apparatus and an underlying surface.

17. The light therapy apparatus of claim 13, wherein when in the inflated state, the base member includes a first width and the shell member includes a second width, and wherein the first width of the base member is greater than the second width of the shell member.

18. The light therapy apparatus of claim 17, wherein when in the inflated state, the shell member includes a dome shape, and the base member includes a flat profile, and wherein the dome shaped shell member is fully disposed on top of the flat profiled base member.

19. The light therapy apparatus of claim 13, wherein the fold line is disposed on the top surface of the base member.

20. The light therapy apparatus of claim 13, wherein the shell member is separable from the base member.
